(19) United States
(12) Patent Application Publication
(10) Pub. No.: US 2014/0038308 A1
(43) Pub. Date: Feb. 6, 2014

(54) PROCESS FOR PRODUCING COMPOSITE DEVICE, AND PROCESS FOR BONDING DEVICE FORMED OF TRANSPARENT MATERIAL TO ADHEREND

(71) Applicant: CANON KABUSHIKI KAISHA, Tokyo (JP)

(72) Inventor: Yoichi Murakami, Tokyo (JP)

(21) Appl. No.: 13/945,206

(22) Filed: Jul. 18, 2013

(30) Foreign Application Priority Data
Aug. 2, 2012 (JP) 2012-171900

(51) Int. Cl. B32B 37/12; G01N 33/50; G01N 33/53; G01N 27/447

(52) U.S. Cl. CPC B32B 37/1284; G01N 27/44791; G01N 33/50; G01N 33/53
USPC 436/501; 156/275.5; 204/451; 436/94

(57) ABSTRACT

Provided is a process for producing a composite device comprising a light shielding first member and a light transmissive second member, a first surface of the light shielding first member and a second surface of the light transmissive second member being bonded to each other through intermediation of an ultraviolet curing adhesive, the second surface being larger than the first surface, the process including irradiating a region of the second surface to which region the first surface is not bonded with an ultraviolet ray, wherein a reflective member having a reflective surface with an inclination with respect to the second surface onto the light transmissive second member so that the ultraviolet ray that has transmitted through the second surface is reflected toward the ultraviolet curing adhesive between the second surface and the first surface.

### BACKGROUND OF THE INVENTION

1. Field of the Invention

The present invention relates to a process for producing a composite device through use of an ultraviolet curing adhesive. The present invention also relates to a process for bonding a device formed of a transparent material to an adherend.

2. Description of the Related Art

In recent years, research and development of a technology called a micro-total analysis system (μ-Tas) in which all elements required for chemical analysis and biochemical analysis are incorporated onto one chip have been actively carried out. Specifically, various devices such as a DNA analysis device, an immunoassay device, and an electrophoresis device have been developed. Each of those devices includes a micro-flow path, a temperature controlling mechanism, a concentration adjusting mechanism, a liquid feeding mechanism, a reaction detecting mechanism, and the like, and is generally called a microfluidic device.

One of the features of the microfluidic device is rapid temperature control. The microfluidic device has a small heat capacity due to a small volume of a minute flow path for controlling temperature, and thus has such a feature that heating and cooling can be performed with a small heat quantity in a short period of time. In order to increase temperature in the micro-flow path, there is a method involving providing a heater in a lower part of the micro-flow path and heating the micro-flow path by the heater to increase the temperature in the micro-flow path. On the other hand, in order to decrease the temperature in the micro-flow path, there is a method involving bonding a heat sink or a Peltier device to the microfluidic device and dissipating heat from a bonding surface by the heat sink or the Peltier device to cool the micro-flow path.

For bonding the microfluidic device to the heat sink or the Peltier device, an adhesive is used for the reasons of ease of operation and high productivity. Adhesives to be used can be roughly classified into three kinds: a cold curing adhesive, a heat curing adhesive, and an ultraviolet curing adhesive. Of those, an ultraviolet curing adhesive has been widely used because time required for bonding can be shortened.

In the microfluidic device, a transparent material that transmits light, such as glass or a silicon-based resin, has been widely used so as to observe the inside of the micro-flow path. On the other hand, an adherend to be bonded to a glass substrate, such as the heat sink or the Peltier device, is formed of a material that does not transmit light, such as aluminum or a printed board. Therefore, when the ultraviolet curing adhesive is used, a composite device is produced by first applying an adhesive onto the microfluidic device and an adherend surface of the adherend, and irradiating the adherend with light from a side of the microfluidic device formed of a transparent material to cure the adhesive through the transparent material, thereby bonding the microfluidic device to the adherend.

However, if a metal film such as a heater is present partially or wholly in the microfluidic device, ultraviolet rays for curing the adhesive are shielded. Therefore, there is a problem in that a part or whole of the adherend surface is not irradiated with ultraviolet rays, with the result that sufficient bonding strength is not obtained.

For example, Japanese Patent Application Laid-Open No. 2003-207790 discloses a method involving curing an adhesive by irradiating a region of a substrate with ultraviolet rays from an oblique direction, the region being not irradiated with ultraviolet rays owing to the presence of a light shielding member even when irradiated with ultraviolet rays from a perpendicular direction.

According to the method of allowing ultraviolet rays to enter the region from an oblique direction, there is a limitation in irradiation depth. The irradiation depth is defined as a distance in an inner direction of the adherend surface with an end of the adherend surface as a reference. The irradiation depth becomes the maximum when ultraviolet rays are allowed to enter a transparent material substantially in parallel with the transparent material and refracted in the transparent material. However, there is a problem in that the entire adherend surface cannot be irradiated with ultraviolet rays when the adherend surface is larger than the maximum irradiation depth.

FIG. 2 illustrates an example thereof. FIG. 2 is a sectional view of a bonded body obtained by bonding a heat sink with a large adherend surface to a microfluidic device by the method involving irradiating a region of a substrate with ultraviolet rays from an oblique direction described in Japanese Patent Application Laid-Open No. 2003-207790. In FIG. 2, a heat sink 21 with an adherend surface having dimensions of 10 mm×10 mm was bonded to a microfluidic device 24 including two glass substrates each having dimensions of 15 mm×30 mm. The microfluidic device 24 includes an upper glass substrate 25 and a lower glass substrate 27. The thickness of each of the upper glass substrate 25 and the lower glass substrate 27 is 500 μm. On a bottom surface of the upper glass substrate 25, a metal film of platinum 29 serving as a heater was formed, patterned, and coated with SiO₂ to a thickness of 2 μm by chemical vapor deposition (CVD). On the lower glass substrate 27, a groove having a depth of 100 μm was formed through use of a resin 26, and the upper glass substrate 25 and the lower glass substrate 27 were bonded to each other to produce a microfluidic device. Then, in order to bond the heat sink 21 and the microfluidic device 24 to each other, the microfluidic device 24 was irradiated with an ultraviolet ray 30 from an oblique direction.

FIG. 3 illustrates an optical path of the ultraviolet ray 30 when the ultraviolet ray 30 is allowed to enter the bonded body illustrated in FIG. 2 from an oblique direction. In order to irradiate an adherend surface 23 with the ultraviolet ray 30, the ultraviolet ray 30 was allowed to enter the adherend surface 23 from an oblique direction. When the incident angle of the ultraviolet ray 30 with respect to the microfluidic device 24 is 90° as illustrated in FIG. 3, the irradiation depth becomes the maximum, and the inside of the adherend surface can also be irradiated with the ultraviolet ray 30. When the refractive index of air is defined as nₐ, the refractive index of the transparent material is defined as n_{b}, and the critical angle is defined as θ_{b}, the critical angle θ_{b} is represented by the following expression according to the Snell's law.

θ_{b}=sin⁻¹(nₐ/n_{b})

When the thickness of the upper glass substrate 25 is defined as D, an irradiation depth 31 in this case is represented by 2D tan θ_{b}. In this case, the distance from an upper surface of the upper glass substrate 25 to the platinum 29 was set to be the same as the thickness of the upper glass substrate 25. Thus, in the case where the adherend surface width W is larger than the irradiation depth, the entire adherend surface 23 cannot be irradiated with the ultraviolet ray 30, and hence sufficient bonding strength cannot be obtained. For example, when the refractive indices of air and glass are 1 and 1.5, respectively, the irradiation depth of FIG. 2 is about 0.9 mm, and accordingly about 90% of the adherend surface 23 is not irradiated with the ultraviolet ray 30. The adherend thus produced was subjected to a breaking test, and the adherend surface 23 was observed. As a result, an uncured portion of an adhesive was recognized on the adherend surface 23.

### SUMMARY OF THE INVENTION

The present invention has been made in view of the above-mentioned related art, and it is an object of the present invention to provide a process for bonding a device formed of a transparent material to a light shielding adherend with a large adherend surface, such as a heat sink or a Peltier device, through use of an ultraviolet curing adhesive.

A production process according to the present invention is a process for producing a composite device including a light shielding first member and a light transmissive second member, a first surface of the light shielding first member and a second surface of the light transmissive second member being bonded to each other through intermediation of an ultraviolet curing adhesive, the second surface being larger than the first surface, the process including irradiating a region of the second surface to which region the first surface is not bonded with an ultraviolet ray, wherein a reflective member having a reflective surface with an inclination with respect to the second surface onto the light transmissive second member so that the ultraviolet ray that has transmitted through the second surface is reflected toward the ultraviolet curing adhesive between the second surface and the first surface.

Further, a bonding process according to the present invention is a process for bonding a device formed of a transparent material to an adherend, the process including: stacking the device and the adherend with an ultraviolet curing adhesive interposed between an upper surface of the device and the adherend; and irradiating the device with an ultraviolet ray substantially perpendicularly from a side of the upper surface, in which, in the inside or on a bottom surface of a region of the device which region is not covered with the adherend and transmits the irradiated ultraviolet ray, at least one reflective member having a reflective surface with an inclination with respect to the bottom surface is provided, and the ultraviolet ray is reflected by the reflective member to be projected to the ultraviolet curing adhesive.

According to one embodiment of the present invention, by irradiating the region of the second surface of the light transmissive member to which region the light shielding member is not bonded with the ultraviolet ray, the ultraviolet curing adhesive under the light shielding member can be cured by diffracted light caused by reflection to bond the light shielding member to the light transmissive member.

Thus, even when a light shielding member such as an electric wire, an electrode pattern, or a flow path is arranged on a side opposite to the second surface of the light transmissive member, and moreover even when a side surface of the light transmissive member is covered with another member, the light shielding member and the light transmissive member can be suitably bonded to each other.

Further, according to the bonding process of the present invention, by reflecting the ultraviolet rays by a reflective member disposed in the inside or on the bottom surface of the device, the ultraviolet ray can be guided to the region that cannot be irradiated with the ultraviolet ray owing to a large adherend surface width even when the ultraviolet ray is allowed to enter the device from an oblique direction, and the adhesive can be cured. Further, in the present invention, the light shielding member and the light transmissive member can be bonded to each other in a short period of time as compared to other bonding processes, for example, using a heat curing adhesive.

Further features of the present invention will become apparent from the following description of exemplary embodiments with reference to the attached drawings.

### BRIEF DESCRIPTION OF THE DRAWINGS

FIG. 1 is a view illustrating an embodiment of the present invention.

FIG. 2 is a sectional view of a bonded body (composite device) obtained by bonding a microfluidic device and a heat sink to each other.

FIG. 3 is a view illustrating an optical path when ultraviolet rays are allowed to enter the bonded body of FIG. 2 from an oblique direction.

FIG. 4 is a sectional view of a bonded body according to Example of the present invention.

FIG. 5 is a diagram showing an angle distribution of ultraviolet rays.

### DESCRIPTION OF THE EMBODIMENTS

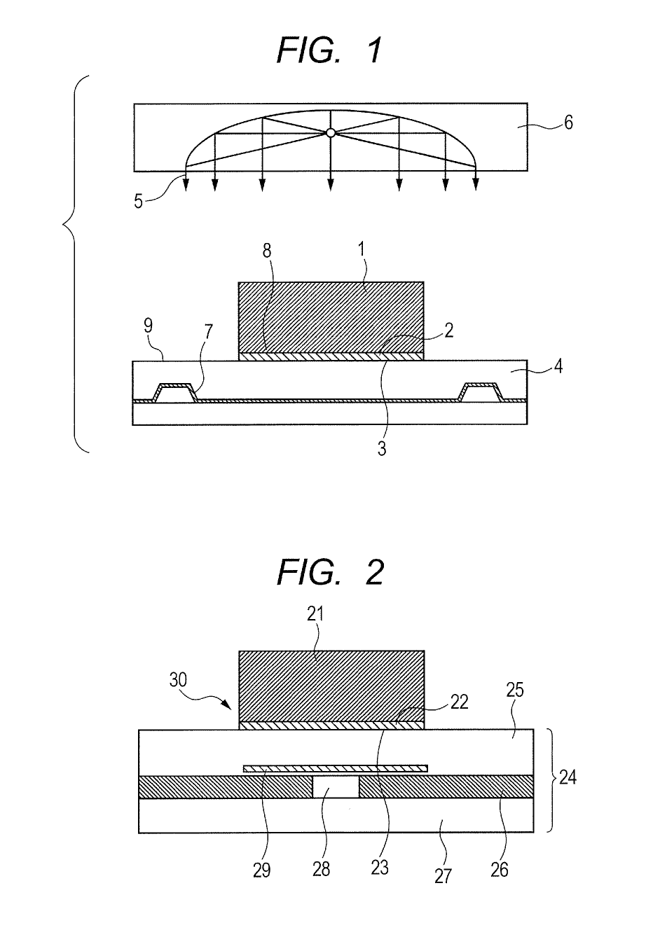

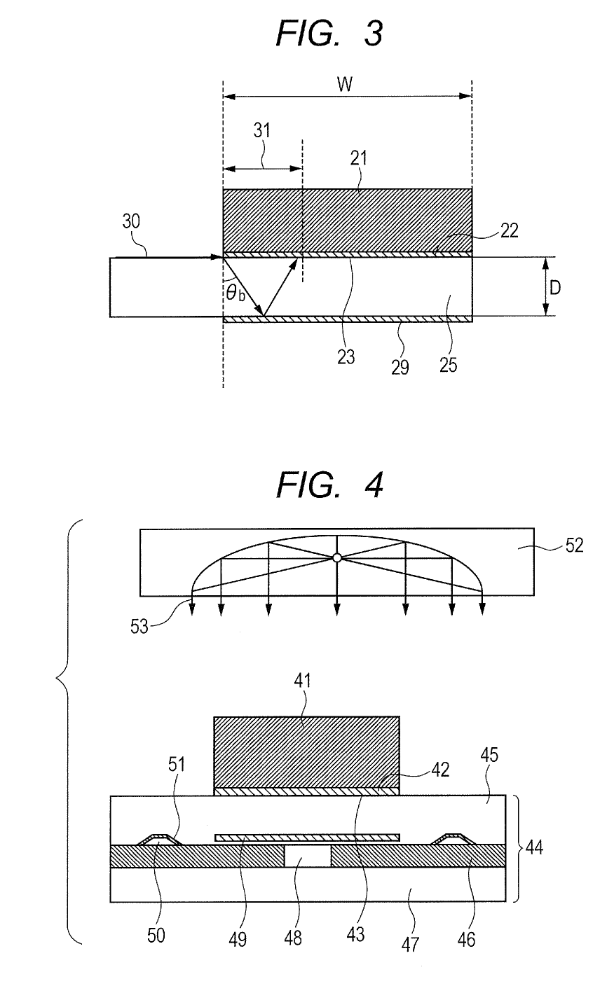

An embodiment of the present invention is a process for bonding a device 4 serving as a second member formed of a light transmissive transparent material to an adherend 1 serving as a light shielding first member, as illustrated in a sectional view of FIG. 1. The device 4 includes a second surface 9 serving as a bonding surface that is an upper surface, and the adherend 1 includes a first surface 8.

The upper surface 9 of the device 4 is larger than the first surface 8 of the adherend 1 and has a region not covered with the adherend 1, that is, a non-bonding surface.

An ultraviolet curing adhesive 2 is applied onto the bonding region of the upper surface 9 of the device 4 with respect to the adherend 1 or onto the first surface 8 of the adherend 1, and the device 4 and the adherend 1 are stacked with the ultraviolet curing adhesive 2 interposed between the upper surface 9 of the device 4 and the adherend 1.

A reflective member 7 having a reflective surface with an inclination with respect to the second surface 9 is provided so that an ultraviolet ray having transmitted through the non-bonding surface of the second surface 9 is reflected toward the ultraviolet curing adhesive 2 between the second surface 9 and the first surface 8.

Accordingly, the ultraviolet curing adhesive 2 can be cured with irradiation light 5 from an illumination member 6 for irradiating the device 4 with an ultraviolet ray substantially perpendicularly from the upper surface side. It is appropriate that, in the inside or on a bottom surface of a region of the device 4 that is not covered with the adherend 1 and transmits the irradiated ultraviolet ray, the reflective member 7 having a reflective surface with an inclination with respect to the bottom surface is provided. The irradiation light 5 is reflected by the reflective member 7, and the reflected light is projected to the adherend surface 3 to cure the ultraviolet curing adhesive 2. Thus, a composite device is produced.

With the above-mentioned configuration, even in the case where the adherend surface width W of the adherend 1 is equal to or more than 2D tan(sin⁻¹(nₐn_{b})) (W≧2D tan(sin⁻¹(nₐ/n_{b}))), where D represents a maximum irradiation depth when an ultraviolet ray is allowed to enter the device 4 from an oblique direction without using reflected light, that is, a distance from the upper surface 9 of the device 4 to a light reflecting surface; nₐ represents a refractive index of air; and n_{b} represents a refractive index of the transparent material, the ultraviolet curing adhesive can be cured.

As the transparent material forming the device 4, a light transmissive material, for example, a plastic resin, a silicone-based resin such as polydimethylsiloxane (PDMS), glass, quartz, or the like is desirably used.

No particular limitation is imposed on the adherend 1 although a device having a temperature adjusting function such as a heat sink or a Peltier device is assumed as the adherend 1.

The illumination member 6 is a metallic illumination unit, and a reflecting mirror having a characteristic of selectively reflecting ultraviolet rays is arranged in the illumination unit. A straight-tube ultraviolet lamp is attached to the inner surface of the reflecting mirror, and a substrate is irradiated with radiation light from the straight-tube ultraviolet lamp substantially perpendicularly directly or after being reflected by the reflecting mirror.

The reflective member 7 is formed of a light reflecting material such as a metal. The reflected light may be projected to the entire adherend surface 1 by only one reflective member 7. Alternatively, as illustrated in FIG. 1, the reflective members 7 may be arranged on both sides of the adherend surface 1 so that the reflected light is projected to the entire adherend surface 1 by the two reflective members 7. Alternatively, the reflected light may be projected to the adherend surface 1 by arranging a plurality of reflective members. Further, the irradiation intensity with respect to the adherend surface 1 may be enhanced by arranging a plurality of reflective members to shorten the time for curing.

### EXAMPLE 1

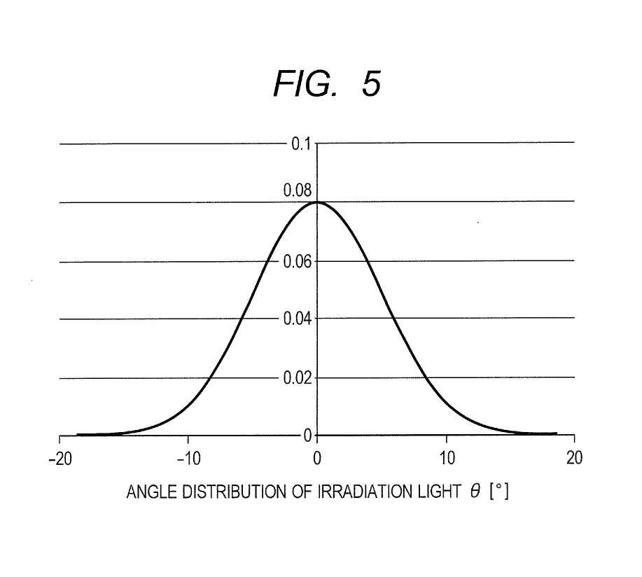

Examples of the present invention are described with reference to FIG. 4. FIG. 4 is a sectional view of a bonded body obtained by bonding a device formed of a transparent material and an adherend to each other through use of an ultraviolet curing adhesive. As the device formed of a transparent material, a microfluidic device 44 formed of two glass substrates having dimensions of 15 mm×30 mm was produced, and as an adherend, a heat sink 41 with an adherend surface 43 having dimensions of 10 mm×10 mm was prepared. The microfluidic device 44 and the heat sink 41 were bonded to each other through use of an ultraviolet curing adhesive 42.

First, a process for producing the microfluidic device 44 is described. The microfluidic device 44 includes an upper glass substrate 45 and a lower glass substrate 47. The thickness of each of the upper and lower glass substrates 45 and 47 is 500 μm. On a bottom surface of the upper glass substrate 45, a metal film of platinum 49 serving as a heater was formed, patterned, and coated with SiO₂ to a thickness of 2 μm by CVD.

Next, the setting positions of a reflective member 51, and the angle (taper angle) of the reflective surface of the reflective member 51 with respect to the bottom surface of the upper glass substrate 45 are described. In this case, the reflective members 51 was arranged on both sides with the adherend surface 43 interposed therebetween, and such an angle and a position of the reflective member 51 that light reflected by the reflective member 51 was projected to the entire adherend surface 43 were calculated. First, the spread of an ultraviolet ray 53 applied from an illumination member 52 was assumed to be a normal distribution of a standard deviation σ of 5° and an average μ of 0°, and the setting position of the reflective member 51 at which the adherend surface 43 was irradiated with the ultraviolet ray 53 with a distribution of −10° to 10° was calculated. FIG. 5 shows an angle distribution (assumed to be a normal distribution) of the ultraviolet ray 53. As a result of the calculation, it was found that, by arranging the reflective member 51 having a reflective surface with a slope angle of 43° and having a depth of 100 μm at a position 2,000 μm outward from an end of the adherend surface 43, the entire adherend surface 43 was irradiated with light having a distribution of −2° to 8°. For example, assuming that the intensity of a light source of the illumination member 52 is 500 [mJ/s·cm²], the transmittance of light when the light enters the upper glass substrate 45 from air is 96%, and the reflectance on the reflective surface is 80%, the intensity of light becomes 385 [mJ/s·cm²]. The irradiation intensity decreases toward the end of the adherend surface 43, and hence the time for curing the ultraviolet curing adhesive 42 at the end of the adherend surface 43 is regarded as the time for curing the entire adherend surface 43. As for the ultraviolet ray 53 with which the end of the adherend surface 43 is irradiated, the end of the adherend surface 43 is irradiated with a total of 2.6 [mJ/s·cm²] of the 8° component (1.6 [mJ/s·cm]) and the −2° component (1.0 [mJ/s·cm²]) reflected by the reflective member 51 set on the opposite side with respect to the adherend surface 43. Assuming that the irradiation amount required for curing the ultraviolet curing adhesive 42 is 500 [mJ/cm²], 190 seconds are required for curing the ultraviolet curing adhesive 42 with this irradiation amount.

Next, the formation of the reflective member 51 is described. A Groove 50 having a taper shape was formed at the above-mentioned setting positions by dry etching. In this case, the taper angle of the groove 50 was adjusted by adjusting the thickness of a resist to be used for dry etching. The taper angle used in this case was set to 43° as described above. After the groove 50 was formed, an Al film was formed on the surface of the groove 50 so as to enhance a reflectance. Further, the Al film was patterned so as to be positioned only inside the groove 50 so as not to prevent the bonding between the upper glass substrate 45 and the lower glass substrate 47.

Next, the formation of a flow path pattern with respect to the lower glass substrate 47 is described. A groove 48 having a depth of 100 μm was formed on the lower glass substrate 47 through use of a resin 46. The upper glass substrate 45 and the lower glass substrate 47 were subjected to plasma irradiation to modify the respective surfaces, and thereafter the upper glass substrate 45 and the lower glass substrate 47 were bonded to each other. Thus, the microfluidic device 44 was produced.

Next, the bonding between the microfluidic device 44 serving as a device and the heat sink 41 serving as an adherend is described. The ultraviolet curing adhesive 42 was applied onto the adherend surface 43. Then, the heat sink 41 was provided so that the platinum pattern of the microfluidic device 44 placed on a stage was covered with the adherend surface 43. Next, the center axis of the main body of the illumination member 52 and the stage were held substantially perpendicularly, and thereafter, the ultraviolet curing adhesive 42 was cured by irradiation with the ultraviolet ray 53 from the illumination member 52 for 190 seconds. The illumination intensity of the ultraviolet ray 53 from the illumination member 52 was set to 500 [mJ/s·cm²], and as the ultraviolet curing adhesive 42, an adhesive requiring an illumination amount of 500 [mJ/cm²] for curing was used. It can be said that the curing time of the ultraviolet curing adhesive 42 is superior to that of a heat curing adhesive requiring a curing time of about 30 minutes.

Finally, the bonded body thus produced was subjected to a breaking test, and the adherend surface 43 was observed. As a result, there was no uncured portion of the ultraviolet curing adhesive 42, and a state in which the ultraviolet curing adhesive 42 was uniformly cured was confirmed.

### EXAMPLE 2

As a modified example of Example 1, the reflective member 51 arranged on the upper glass substrate 45 in FIG. 4 may be arranged on the lower glass substrate 47. For example, a reflective member can be formed by forming a groove serving as the reflective member simultaneously with the formation of the groove 48 on the lower glass substrate 47 and forming an Al film on the groove serving as the reflective member.

The composite device obtained by the production process of the present invention can be used as a so-called microfluidic composite device by additionally providing a micro-flow path, a temperature controlling mechanism, a concentration adjusting mechanism, a liquid feeding mechanism, a reaction detecting mechanism, and the like.

That is, a liquid containing an analyte or the like can be allowed to flow through the flow path for chemical analysis or biochemical analysis. Specifically, the composite device of the present invention can be applied to various devices such as a DNA analysis device, an immunoassay device, and an electrophoresis device.

The composite device of the present invention can control temperature rapidly and has a small heat capacity due to a small volume of a minute flow path for controlling temperature. Thus, heating and cooling can be performed with a small heat quantity in a short period of time.

In order to increase the temperature in the micro-flow path, it is appropriate that a heater is provided in a lower part of the micro-flow path and the micro-flow path is heated by the heater to increase the temperature in the micro-flow path. On the other hand, in order to decrease the temperature in the micro-flow path, it is appropriate that a heat sink or a Peltier device serving as a first member is bonded to the composite device and heat is dissipated from an adherend surface by the heat sink or the Peltier device to cool the micro-flow path.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2012-171900, filed Aug. 2, 2012, which is hereby incorporated by reference herein in its entirety.

What is claimed is:

1. A process for producing a composite device comprising a light shielding first member and a light transmissive second member, a first surface of the light shielding first member and a second surface of the light transmissive second member being bonded to each other through intermediation of an ultraviolet curing adhesive, the second surface being larger than the first surface,
the process comprising irradiating a region of the second surface to which region the first surface is not bonded with an ultraviolet ray,
wherein a reflective member having a reflective surface with an inclination with respect to the second surface onto the light transmissive second member so that the ultraviolet ray that has transmitted through the second surface is reflected toward the ultraviolet curing adhesive between the second surface and the first surface.

2. A process for producing a composite device according to claim 1, wherein the composite device has a groove to be a flow path on a side of a surface of the second member which surface is opposite to the second surface.

3. A process for producing a composite device according to claim 1, wherein the composite device has a metal film formed on a side of a surface of the second member which surface is opposite to the second surface.

4. A process for producing a composite device according to claim 1, wherein the step of irradiating the region of the second surface with the ultraviolet ray comprises irradiating the region of the second surface with the ultraviolet ray substantially perpendicularly.

5. A process for producing a composite device according to claim 1, wherein an adherend surface width W of the first member satisfies W≧2D tan(sin⁻¹(nₐ/n_{b}) where D represents a distance from the second surface of the second member to a light reflecting surface, nₐ represents a refractive index of air, and n_{b} represents a refractive index of the second member.

6. A process for producing a composite device according to claim 1, wherein the first member comprises a heat sink.

7. A process for producing a composite device according to claim 1, wherein the second member comprises a microfluidic device.

8. A process for performing one of chemical analysis and biochemical analysis through use of a composite device produced by the process for producing a composite device according to claim 7.

9. A process for bonding a device formed of a transparent material to an adherend,
the process comprising:
stacking the device and the adherend with an ultraviolet curing adhesive interposed between an upper surface of the device and the adherend; and
irradiating the device with an ultraviolet ray substantially perpendicularly from a side of the upper surface,
wherein, in the inside and or on a bottom surface of a region of the device which region is not covered with the adherend and transmits the irradiated ultraviolet ray, at least one reflective member having a reflective surface with an inclination with respect to the bottom surface is provided, and the ultraviolet ray is reflected by the reflective member to be projected to the ultraviolet curing adhesive.

10. A process for bonding a device formed of a transparent material to an adherend according to claim 9, wherein an adherend surface width W of the adherend satisfies W≧2D tan(sin⁻¹(nₐ/n_{b})) where D represents a distance from the upper surface of the device to a light reflecting surface, nₐ represents a refractive index of air, and n_{b} represents a refractive index of the transparent material.

11. A process for bonding a device formed of a transparent material to an adherend according to claim 9, wherein the device comprises a microfluidic device.
